(19) United States
(12) Patent Application Publication
(10) Pub. No.: US 2024/0336884 A1
(43) Pub. Date: Oct. 10, 2024

(54) BIOREACTOR SYSTEM WITH EXTERNAL LOOP FOR ENHANCED GAS TRANSFER AND/OR TRANSFECTION

(71) Applicant: UNIVERCELLS TECHNOLOGIES SA, Nivelles (BE)

(72) Inventors: Jean-Christophe Drugmand, Wavre (BE); Felix Benjamin Kasper, Nivelles (BE); Tania Pereira Chilima, Nivelles (BE)

(21) Appl. No.: 18/293,117

(22) PCT Filed: Jul. 28, 2022
(86) PCT No.: PCT/EP2022/071246

§ 371 Date: Jan. 29, 2024

Related U.S. Application Data
(60) Provisional application No. 63/226,372, filed on Jul. 28, 2021.

(51) Int. Cl. C12M 1/12; C12M 1/00; C12M 1/34

(52) U.S. Cl. CPC C12M 25/18; C12M 29/06; C12M 29/18; C12M 41/34

(57) ABSTRACT

An apparatus is provided for culturing and transfecting cells using a liquid. A bioreactor includes a cell culture bed for receiving the liquid. An external loop is connected to the bioreactor and adapted for circulating the liquid to and from the bioreactor. The external loop is adapted for introducing a gas into the liquid therein and/or for circulating a transfection material in the liquid therein to the bioreactor. In one embodiment, a first vessel is associated with the external loop for receiving the liquid, and a gas injector is provided for injecting a gas into the liquid, such as into the first vessel. A second vessel is also provided for providing a transfection material (such as a transfecting agent as part of a transfection mixture) to the liquid, such as directly to the bioreactor or to the external loop. In another embodiment, the gas injector is associated directly with the external loop. The external loop may be dedicated for gas injection and/or transfection, and maintained separate from a loop for circulating liquid media to the bioreactor, which potentially allows for operation on demand. Related methods are also disclosed.

This application claims the benefit of U.S. Provisional Patent Application Ser. No. 63/226,372, filed Jul. 28, 2021, the disclosure of which is incorporated herein by reference, and also incorporates by reference International Patent Application PCT/EP2020/084317, U.S. Provisional Patent Application Ser. Nos. 62/758,152, 62/733,375, 62/608,261, 63/004,706, and 62/942,345; U.S. Patent Application Publication No. 2018/0282678; International Patent Application PCT/EP2018/076354; U.S. Provisional Patent Application 62/711,070, and U.S. Provisional Patent Application 62/725,545.

### TECHNICAL FIELD

This document relates generally to the cell culturing arts and, more particularly, to a bioreactor with an external loop for enhanced gas transfer and/or transfection.

### BACKGROUND

Bioreactors are frequently used for culturing organisms, such as cells, bacteria, yeast, or the like. Within the bioreactor, a certain level of gas transfer between the gas phase (e.g. air or oxygen) and the liquid phase (the cell culture medium) is required to permit optimal respiration for cell growth. A supply of oxygen to the cells is mandatory in such gas transfer.

Gas transfer is especially important for a fixed-bed bioreactor for animal cells, the high cell density growth of which demands a heightened level of oxygen. The amount of oxygen present for enhancing cell growth may be assessed using the characterization of gas transfer coefficient, or kLa. This value uses the volumetric mass-transfer coefficient that describes the efficiency with which gas can be delivered to a bioreactor for a given set of operating conditions. Gas transfer may also include stripping gas (such as carbon dioxide produced by the growing cells) from the bioreactor during use.

In the past, proposals have been made for enhancing the gas transfer by providing the bioreactor with one or more bubblers or spargers to form small gas bubbles in the cell culture medium. However, when a sparger is used to support a high cell density bioreactor, the speed of bubble input and the need for an extensive amount of bubbles often results in the negative effects of shear and excessive foaming. This can deleteriously cause undue mechanical and oxidative stress to the culture media and the cells, which can impact yields. For instance, foam can also damage the protein of the culture media or make them not available anymore for the cells if the proteins are trapped into the foam, and thereby decrease the yield. Further, foaming can clog filters and other connectors in the bioreactor. In addition, use of spargers in unstructured high cell density fixed bed bioreactors is challenging, as the resulting bubbles can cause large “clouds” or air pockets as the bubbles have no definable path through the fixed bed. Such bubbles negatively affect homogeneity of the bioreactor conditions and, thus, cell growth.

In addition to cell growth, bioreactors may also be used for transfection, which is the process of artificially introducing nucleic acids (DNA or RNA) into cells, utilizing means other than viral infection. A large volume of transfection material (e.g., transfection agent and/or plasmid) required to be added for some gene therapy processes could exceed the volume of the relatively smaller fixed bed bioreactor. While transfection can be accomplished by emptying and refilling the bioreactor with stopping of the agitation, such methodology includes the risk of having a deleterious non-homogeneous transfection.

Accordingly, a need is identified for a bioreactor, particularly a high cell density fixed bed bioreactor, that may provide enhanced gas transfer and also be adapted for use in connection with transfection, such as by providing one or more vessels associated with an external loop connected to the bioreactor for gas transfer, transfection, or both independently.

### SUMMARY

An object of this disclosure is to provide a bioreactor, particularly a high cell density fixed bed bioreactor, with enhanced gas transfer and for use in connection with transfection, such as by providing one or more vessels associated with an external loop connected to the bioreactor for gas transfer, transfection, or both independently.

According to one aspect of the disclosure, an apparatus for culturing and transfecting cells using a liquid includes a bioreactor including a cell culture bed for receiving the liquid. A first external loop is connected to the bioreactor for circulating the liquid. A first vessel is associated with the external loop for receiving the liquid. A gas injector is provided for injecting a gas into the liquid. A second vessel may also provide a transfection material to the liquid.

In one embodiment, the second vessel is adapted for delivering the transfection material directly to the bioreactor. In another embodiment, the second vessel is adapted for delivering the transfection material or other reagents for gene editing directly to the external loop. In any embodiment, the gas injector is adapted for delivering gas to the first vessel, to the first external loop, or both. A second external loop may also be provided for circulating liquid from a second vessel, such as a liquid media supply tank, to the bioreactor.

To further enhance gas transfer, the bioreactor may further include a sparger. Alternatively or additionally, the bioreactor is adapted for forming a falling liquid to promote gas exchange therein. The bioreactor, the first vessel, and the second vessel may each include an agitator. The first external loop includes one or more pumps, as may the second external loop if present. The one or more pumps may comprise one or more low-shear pumps.

The apparatus may further include a sensor for sensing an amount of gas in the liquid. The sensor generates an output signal for use in regulating one or more pumps for pumping liquid through the first external loop or regulating the gas injector for injecting gas into the liquid. The sensor may be adapted for sensing the amount of gas within the liquid in the bioreactor, and may comprise for example a dissolved oxygen sensor (which may be associated with the bioreactor or an external loop).

The first external loop may include a mixer, such as a static mixer. The cell culture bed may comprises a structured fixed bed, a spiral bed, or a 3-D printed monolithic matrix. In any embodiment, the transfection material may be a component of a transfecting mixture added to the liquid.

According to a further aspect of the disclosure, an apparatus for culturing and transfecting cells using a liquid is provided. The apparatus includes a bioreactor including a cell culture bed for receiving the liquid. A first external loop is connected to the bioreactor and adapted for circulating the liquid via one or more low shear pumps. A gas injector is provided for injecting a gas into the liquid. A first vessel is for supplying a transfection material to the liquid.

In one embodiment, the first vessel is adapted for delivering the transfection material directly to the bioreactor. Alternatively, the first vessel may be adapted for delivering the transfection material directly to the first external loop, or to both the bioreactor and/or vessel. The delivery of the transfection material may occur in different stages or at different times.

In these or other embodiments, the gas injector is adapted for delivering gas to the external loop, to a second vessel connected to the external loop, or both. A second external loop may be provided for circulating liquid from a tank or like vessel to the bioreactor. The bioreactor may further include a sparger and/or may be adapted for forming a falling liquid to promote gas exchange therein. The bioreactor and the vessel may each include an agitator.

In any embodiment, a sensor is provided for sensing an amount of gas in the liquid. The sensor generates an output signal for use in regulating the one or more low shear pumps for pumping liquid through the first external loop or regulating the gas injector. The sensor may be adapted for sensing the amount of gas within the liquid in the bioreactor, and may comprise a dissolved oxygen sensor.

In any embodiment, the first external loop includes a mixer, such as a static mixer. The cell culture bed may comprise a structured fixed bed, a spiral bed, or a 3-D printed monolithic matrix.

According to yet another aspect of the disclosure, an apparatus for culturing cells using a liquid is provided. The apparatus comprises a bioreactor including a cell culture bed for receiving the liquid. A first external loop is connected to the bioreactor and adapted for circulating the liquid, the first external loop adapted for introducing a gas into the liquid therein or for circulating a transfection material in the liquid therein to the bioreactor. A media vessel is provided for supplying the liquid to the bioreactor independent of the first external loop, such as by a direct connection to the bioreactor, or via an optional second external loop.

In one embodiment, the first external loop comprises a gas injector. The gas injector may comprise a sparger associated with a vessel, the vessel optionally associated with a mixer. The gas injector may comprise a gas line for supplying the gas directly to the first external loop, which may include one or more pumps (such as low shear pumps for example). The first external loop includes one or more low-shear pumps, and further including a vessel for introducing a transfection material to the cell culture bed.

A sensor may also be provided for sensing an amount of gas in the liquid. The sensor generates an output signal for use in regulating one or more pumps for pumping liquid through the first external loop or the gas injector. The first external loop may include a mixer. The cell culture bed may comprise a structured fixed bed, a spiral bed, or a 3-D printed monolithic matrix.

Still a further aspect of the disclosure pertains to an apparatus for culturing cells using a liquid. The apparatus comprises a bioreactor including a cell culture bed for receiving the liquid. At least one gas sensor is for sensing an amount of gas in the liquid. An external loop is connected to the bioreactor, the external loop associated with a pump for circulating the liquid and a gas injector for injecting a gas into the liquid. A controller is for controlling the pump, the gas injector, or both, based on the output of the at least one gas sensor.

In one embodiment, the gas injector comprises a sparger associated with a vessel, optionally associated with a mixer. The gas injector may comprise a gas line for supplying the gas directly to the external loop. The external loop may include a plurality of pumps, and may connect to an inlet and an outlet of the bioreactor. The external loop may include a static mixer. The cell culture bed may comprise a structured fixed bed, a spiral bed, or a 3-D printed monolithic matrix.

In accordance with still another aspect of the disclosure, an apparatus for culturing cells using a liquid includes a bioreactor including a cell culture bed for receiving the liquid, the bioreactor adapted for promoting gas exchange with the liquid therein. An external loop is connected to the bioreactor, the external loop associated with a pump for circulating the liquid. A gas injector is provided for injecting a gas into the liquid, such as via the external loop, an external vessel, or both. The bioreactor may be adapted for creating a falling film of liquid in a headspace thereof.

Yet another aspect of the disclosure pertains to a method for culturing and transfecting cells using a bioreactor including a cell culture bed for receiving a liquid media from a media source. The method comprises introducing a transfection material to the liquid media. The method further comprises circulating the liquid media including the transfection material via an external loop connected to the bioreactor and separate from the media source.

The introducing step may comprise introducing a transfection mix including the transfection material to the bioreactor prior to the circulating step, or to the external loop. The method may further include the step of halting any delivery of liquid media from the media source during the introducing and circulating steps. The method may also include the step of injecting gas into the liquid via the external loop prior to the introducing step, such as into a vessel connected to the external loop and/or directly to the external loop. The method may include the step of connecting the external loop to the bioreactor after cell culturing is complete and prior to the introducing step.

A further aspect of this disclosure relates to a method for regulating the culturing of cells using a bioreactor including a cell culture bed for receiving a liquid from a media source. The method comprises injecting a gas into a liquid in an external loop connected to the bioreactor and independent of the media source. The gas injecting step may comprise injecting the gas into a vessel connected to the external loop and/or the gas directly to the external loop. The method may further comprise halting the gas injecting step, introducing a transfection material to the liquid, and circulating the liquid including the transfection material via the external loop. Still further, the method may include regulating the injecting of the gas based on a sensed parameter of the liquid, and/or regulating a flow rate of the liquid in the external loop based on a sensed parameter.

It is another aspect of this disclosure to provide a method for regulating the culturing of cells using a bioreactor including a cell culture bed for receiving a liquid from a media source. The method comprises sensing a parameter of the liquid. Based on the sensed parameter, the method comprises regulating the delivery of liquid within an external loop connected to the bioreactor, the external loop adapted for injecting gas to the liquid, regulating the injecting of gas to the external loop, or both. The method further includes the step of injecting gas into the liquid via the external loop and/or injecting the gas into a vessel connected to the external loop. The sensing step may comprise dissolved oxygen as the liquid, which may be completed by a sensor in the bioreactor and/or a sensor in the external loop.

A related aspect of this disclosure is a method of bioprocessing including culturing cells in a bioreactor including a liquid. During the culturing step, gas is injected into the liquid in an external loop in fluid communication with the bioreactor. The method further comprises transfecting the cells by adding a transfection material to the liquid, and delivering a portion of the liquid including the transfection material to the external loop.

In one embodiment, the external loop includes a vessel, and the injecting step comprises injecting gas into the vessel. The delivering step may also comprise delivering the portion of the liquid including the transfection material to the external loop. The transfecting step may comprise adding the transfection material to the bioreactor.

The method may further include the step of sensing a parameter of the liquid. Based on the sensed parameter, the method may regulate the delivery of liquid to the external loop or the injecting of gas to the external loop.

The transfecting step may comprise circulating the transfecting material within the bioreactor for a predetermined time. The method may further include the step of halting the gas injecting step prior to and during the transfecting step.

### BRIEF DESCRIPTION OF THE DRAWING FIGURES

FIG. 1 is a perspective view of an exemplary bioreactor for which certain aspects of this disclosure may have applicable;

FIG. 2 is a partially exploded view of further details of the bioreactor of FIG. 1;

FIG. 3 illustrates a wound or “spiral” fixed bed for possible use in connection with a bioreactor;

FIGS. 3A, 3B, and 3C illustrate particular details of the spiral fixed bed;

FIGS. 3D, 3E and 3F illustrate alternative arrangements for forming a structured fixed bed;

FIG. 4 illustrates another form of structured fixed bed;

FIG. 5 illustrates a first embodiment of a bioreactor including an external fluid flow or “loop”;

FIGS. 6, 6A, and 6B illustrate a second embodiment of a bioreactor including an external loop;

FIG. 7 is a schematic diagram illustrating a process control approach to regulating gas transfer to a liquid in an external loop based on sensed demand;

FIG. 8 is a flow chart related to the process control of FIG. 7;

FIG. 9 illustrates a further embodiment of a bioreactor including an external loop associated with a vessel including a transfection material;

FIG. 10 illustrates the substitution of different vessels into an external loop for either enhanced gas transfer or transfection; and

FIG. 11 is a flow chart illustrating the possible steps of performing transfection using an external loop.

### DETAILED DESCRIPTION

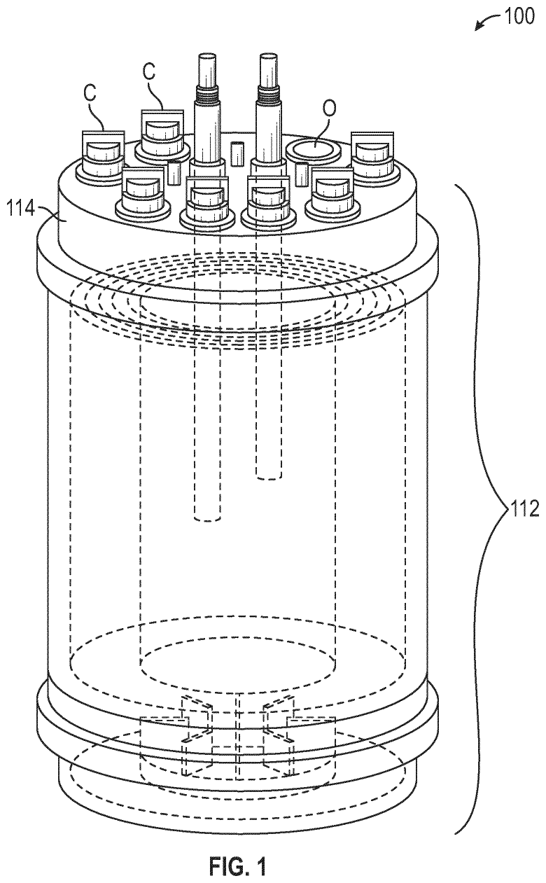

Reference is now made to FIGS. 1-3, which illustrate one embodiment of a bioreactor 100 for culturing cells according to one aspect of the disclosure. The bioreactor 100 may include an external casing or housing 112 forming an interior compartment and an optional cover 114 is placed on top of the housing to cover or seal the interior compartment after it is populated with at least a cell culture bed. In an embodiment, the cover is removable. The cover 114 may include various openings or ports O with removable closures or caps C for allowing for the selective introduction or removal of material, fluid, gas, probes, sensors (including for example a dissolved oxygen sensor), samplers, or the like.

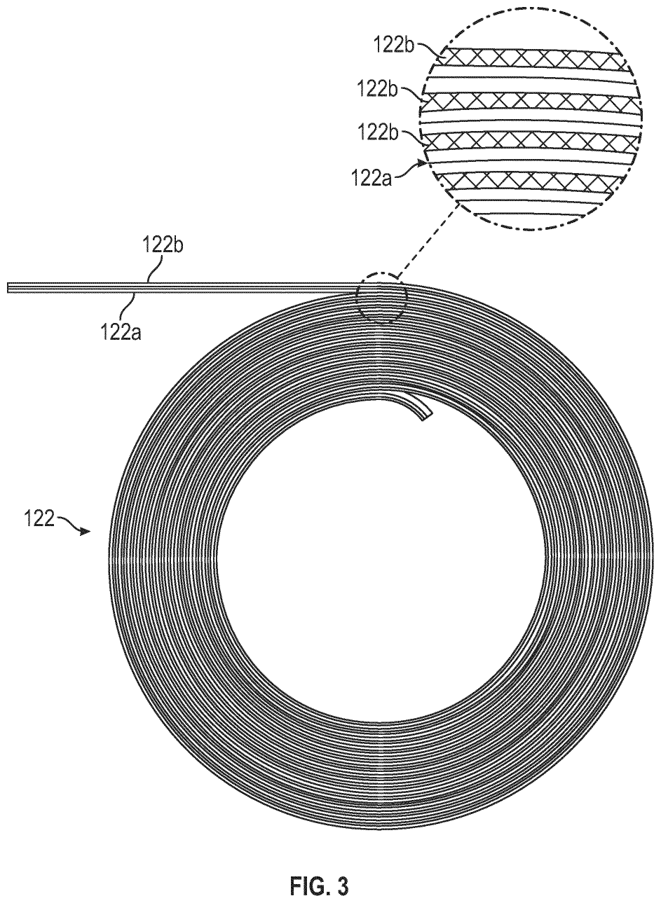

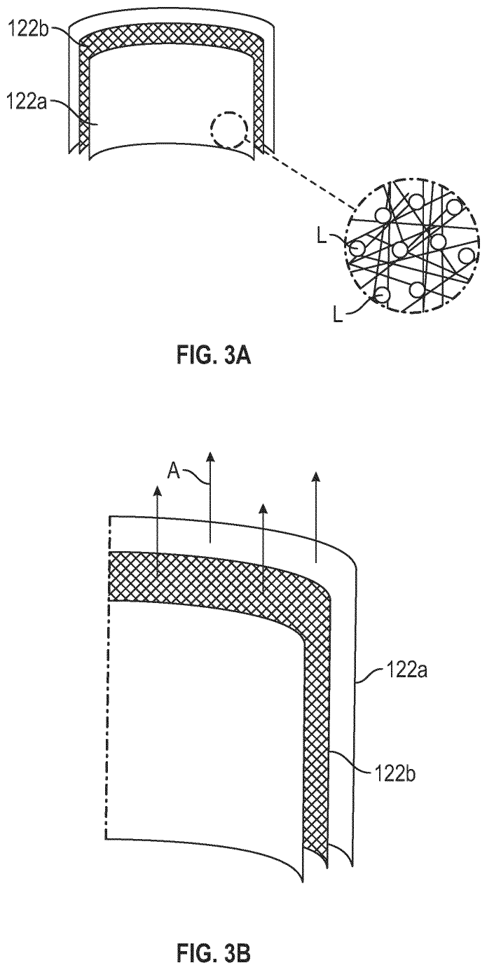

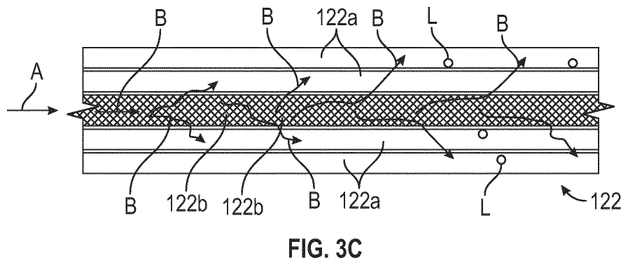

Within the interior compartment formed by the bioreactor housing 112, several compartments or chambers may be provided for transmitting a flow of fluid, gas, or both, throughout the bioreactor 100. As indicated in FIG. 3, the chambers may include a first chamber 116 at or near a base of the bioreactor 100. In some embodiments, the first chamber 116 may include an agitator for causing fluid flow within the bioreactor 100. In some embodiments, the agitator may be in the form of a “drop-in” rotatable, non-contact magnetic impeller 118, which thus forms a centrifugal pump in the bioreactor. The agitator could also be in the form of an impeller with a mechanical coupling to the base, an external pump forming part of a fluid circulation system, or any other device for causing fluid circulation within the bioreactor.

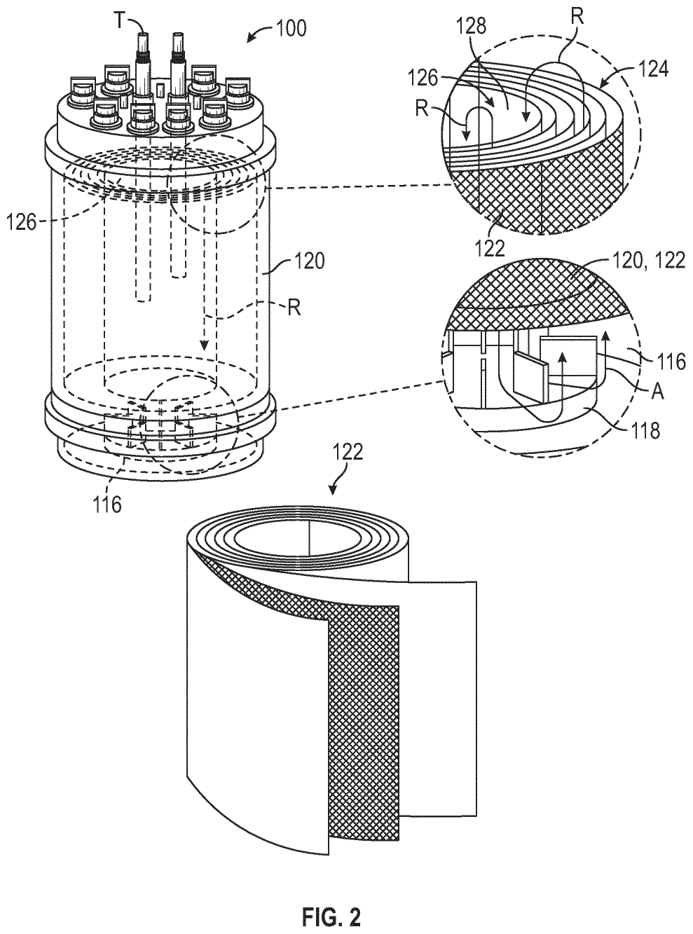

As a result of the agitation provided, fluid may then flow upwardly (as indicated by arrows A in FIG. 2) into a chamber 120 along the outer or peripheral portion of the bioreactor 100. In some embodiments, the bioreactor is adapted to house a cell culture bed in any form including a packed bed, fixed bed, a structured fixed bed, a fluidized bed, etc. FIG. 3 shows a fixed bed in the form of a structured spiral bed 122 which, in use, may contain and retain cells being grown. In some embodiments, the spiral bed 122 may be in the form of a cartridge that may be dropped or placed into the chamber 120. The bed 122 can be pre-installed in the chamber during manufacture at a facility prior to shipping or installed at the point of use.

Fluid exiting the chamber 120 is passed to a headspace formed by a chamber 124 on one (upper) side of the bed 122, where the fluid is exposed to a gas (such as oxygen). Fluid may then flow radially inwardly to a central chamber 126 to return to the lower portion of bed 122. In some embodiments, this central chamber 126 can be columnar in nature and may be formed by an imperforate conduit or tube 128 or rather formed by the central opening of the structured bed.

The chamber 126 returns the fluid to the first chamber 116 (return arrow R) for recirculation through the bioreactor 100, such that a continuous loop results (“bottom to top” in this version). This fluid in returning may form a falling film, which promotes gas exchange, and may eliminate the need for including a sparger in the bioreactor 100, which can sometimes lack suitable space for such, and in any case avoids the added expense and complexity. In some embodiments, a sensor, for example a temperature probe or sensor T may also be provided for sensing the temperature of the fluid in the chamber 126. In some embodiments, sensors (such as, for example, pH, oxygen, dissolved oxygen, temperature, cell density, etc.) may also be provided at a location before the fluid enters (or re-enters) the chamber 116.

FIG. 3 shows one embodiment of a matrix material for use as a structured fixed bed in the bioreactor of the present disclosure and, in particular, a wound or spiral bed 122. In some embodiments, one or more cell immobilization layers 122a are provided adjacent to one or more optional spacer layers 122b made from a woven or non-woven mesh structure. In some embodiments, the layering may optionally be repeated several times to achieve a stacked or layered configuration.

The mesh structure included in spacer layers 122b forms a tortuous path for cells (see cells L in FIG. 3A suspended or entrapped in the material of the immobilization layer 122a), and a cell culture may form part of any invention claimed herein) and fluid to flow through a channel thus created when layered between two immobilization layers 122a. Homogeneity of the cells is maintained within the structured fixed bed as a result of this type of arrangement. In some embodiments, other spacer structures can be used which form such tortuous paths.

As shown in FIGS. 3, 3A and 3B, the structured fixed bed can be subsequently spirally or concentrically rolled along an axis or core (e.g., conduit or tube 128, which may be provided in multiple component parts). In some embodiments, the layers of the structured fixed bed are firmly wound. In some embodiments, the diameter of the core, the length and/or amount of the layers will ultimately define the size of the assembly or matrix. In some embodiments, thickness of each of the layers 122a, 122b may be between 0.1 and 5 mm, 0.1 and 10 mm, or 0.001 and 15 mm.

In some embodiments, other structures can be used which form such tortuous paths. For example, FIG. 3D shows that the one or more cell immobilization layers 122a may be adapted to form a structured fixed bed 122. The one or more layers 122a provide a tortuous channel of flow (arrow B) from a linear or regular inflow (arrow A) without using additional spacer layers (but such may be used, if desired). This may be achieved, for example, by providing one or more layers of woven fibers or filaments 123, 125 that disrupt the flow.

FIG. 3E shows that such a result may be achieved using a non-woven material as the cell immobilization layer 122a. This may be achieved by forming the layer 122a as a reticulated arrangement (such as by 3-D printing) with openings 127 through which liquid may pass and return again, thus forming the tortuous channels that again promote homogeneity and also serve to further shear or divide any bubbles present in the liquid. This function may again be achieved with or without added spacer layers being present.

The orientation of the structured fixed bed 122 may be other than as shown in a bioreactor 100 as shown in FIG. 2, where the flow is arranged vertically (bottom to top, in the example provided). For example, as shown in FIG. 3F, a bioreactor 100 may include a first chamber 120 that includes a structured fixed bed 122 comprised of one or more horizontally arranged material layers. The one or more layers may comprise a woven or reticulated material, as per FIGS. 3D and 3E, but as illustrated in FIG. 3F, may comprise one or more cell immobilization layers 122a (three shown, but any number may be present) sandwiched by adjacent spacer layers 122b (vertical spacing exaggerated for purposes of illustration), which are optional. The flow is thus arranged from side-to-side (left to right or right to left), with the material layer(s) (spacer or otherwise) providing for the channels for creating the tortuous flow (arrows B) from a linear or regular inflow (arrow A) and thus serving to further divide any bubbles present in the liquid. The pumping action may be provided by an agitator or other pump at the entrance end of the chamber 120, and a return path provided at the exit end, as schematically illustrated by path R. Additional spacer layers may optionally be provided between the cell immobilization layers 122a.

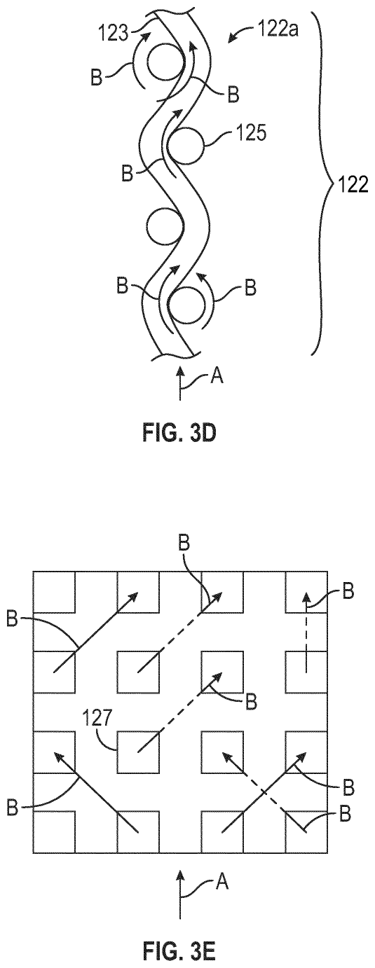

In another possible embodiment, and with reference to FIG. 4, the structured fixed bed 122 comprises a three-dimensional (3D) monolith matrix 124 in the form of a scaffold or lattice formed of multiple interconnected units or objects 124a, which have surfaces for cell adhesion. Preferably, the matrix includes a tortuous path for fluid and cells to flow therethrough when in use. In some embodiments, the matrix may be in the form of a 3D array, lattice, scaffolding, or sponge, and can be in any shape (e.g., cylindrical, cubic, etc.). The matrix 124 is preferably single use in nature to avoid the cost and complexities involved in cleaning according to bioprocessing standards.

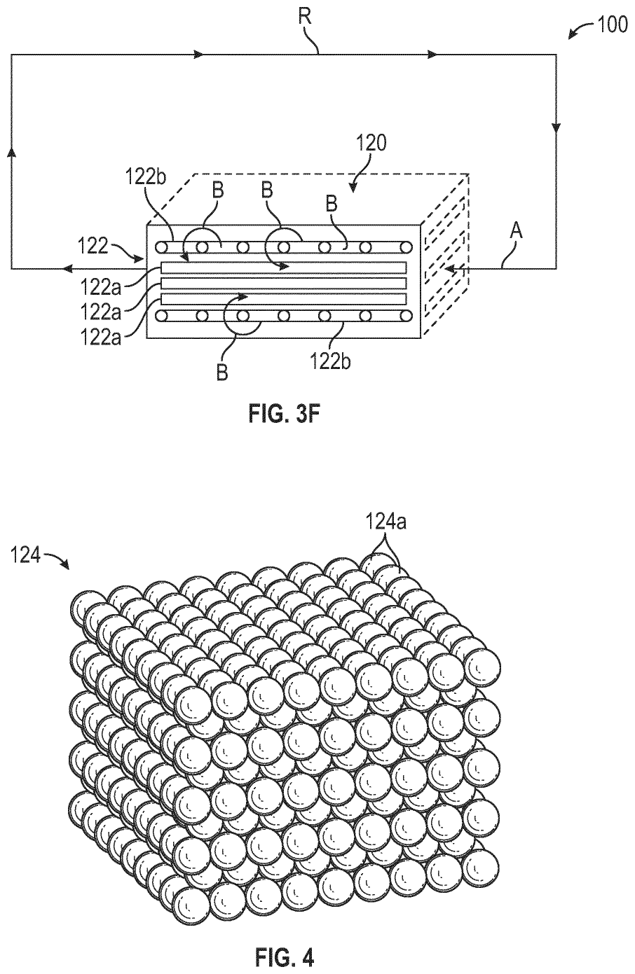

As noted previously, it may be desirable to increase the amount of gas transfer to the liquid for culturing cells in the bioreactor 100, as well as to enhance the ability to strip or remove unwanted gases from the liquid (such as, for example, carbon dioxide). According to one aspect of the disclosure, and with reference to FIG. 5, this may be achieved by introducing a flow of gas, such as air or oxygen, into a dedicated external fluid circulation loop 200 for transmitting liquid to and from the bioreactor 100. The external loop 200 may include an input end 202 located in the bioreactor 100 for drawing out liquid.

The bioreactor 100 may also be associated with a gas inlet 103, which may be used to supply gas (e.g., air, carbon dioxide (such as for pH regulation), or oxygen). The gas delivery may be directly to the bioreactor, such as in a headspace thereof, and/or in connection with a sparger at least partially submerged in the liquid. The bioreactor 100 may optionally include a vent 105 for use in releasing gas from the bioreactor.

A vessel 206 forming part of the loop 200 receives the withdrawn liquid from a first portion 200a (e.g., in the form of a suitable conduit or tubing) of the loop 200, such as by using a pump 204 in line with the portion 200a. The vessel 206 may comprise a rigid or flexible vessel (including possibly a disposable or single-use vessel), such as a bladder or bag. This vessel 206 may include a gas injector, such as for example a sparger 208 (such as, for example, a microsparger, which may comprise a sparger pipe and a porous frit optionally made of metal, such as stainless steel). The gas injector or sparger 208 may receive a supply of gas, such as air or oxygen, from an external source, such as a tank 210 or the environment. The vessel 206 may also comprise a bubble column. However, as noted above, it is possible for the gas transfer to be enhanced simply via the headspace in the vessel 206. The vessel 206 may also be considered a gas introduction vessel or gas introduction system.

In any case, the vessel 206 may be associated with a mixer, such as an internal agitator (e.g., a stir bar coupled via a non-contact (magnetic) coupling to an external drive) or an external orbital shaker 212. The resulting agitation helps mix the gas and liquid and thereby enhance gas transfer to the liquid. The vessel 206 may also include a suitable vent 214 for venting gas to the atmosphere, which may be useful in situations where stripping of gas is desired (any suitable device for pulling gas out of fluid may be used). The arrangement may be so as to maintain the aseptic condition of the liquid during the gas transfer process such as by using aseptic connectors or otherwise.

The gas-enhanced liquid may then be returned from the vessel 206 to the bioreactor 100, such as via a second portion 200b (e.g. in the form of a suitable conduit or tubing) of the loop 200. This may be achieved using a second pump 216 in line with the portion 200b. An output end 218 of the loop 200 may communicate directly with the bioreactor 100, or may admix the liquid from the vessel with liquid being introduced to the bioreactor, or perfusate, via a suitable conduit 102 serving as inlet and associated with a pump 106 and an upstream source (not shown). A suitable conduit 104 associated with a pump 108 may deliver liquid from the bioreactor to a harvest vessel or other downstream process, or instead may be delivered to a media tank for recirculation via a conduit distinct from the external loop 200 for enhanced gas transfer.

In any case, the ability to enhance the gas transfer using an external loop 200 dedicated for this purpose may avoid the above-mentioned issues that sometimes arise from associating a sparger with the bioreactor 100, and especially one having a fixed bed 122 with a high cell density. This dedicated external loop 200 also operates completely independently of any liquid media delivery to the bioreactor 100, and thus may be used on demand, if desired, without impacting the regular flow of media to or from the bioreactor. Furthermore, enhancing the gas transfer in an external vessel may avoid the foaming associated with the gas exchange process when completed in the bioreactor 100, and thus facilitate the inclusion of the above-mentioned sensors directly in the bioreactor for sensing conditions without becoming fouled.

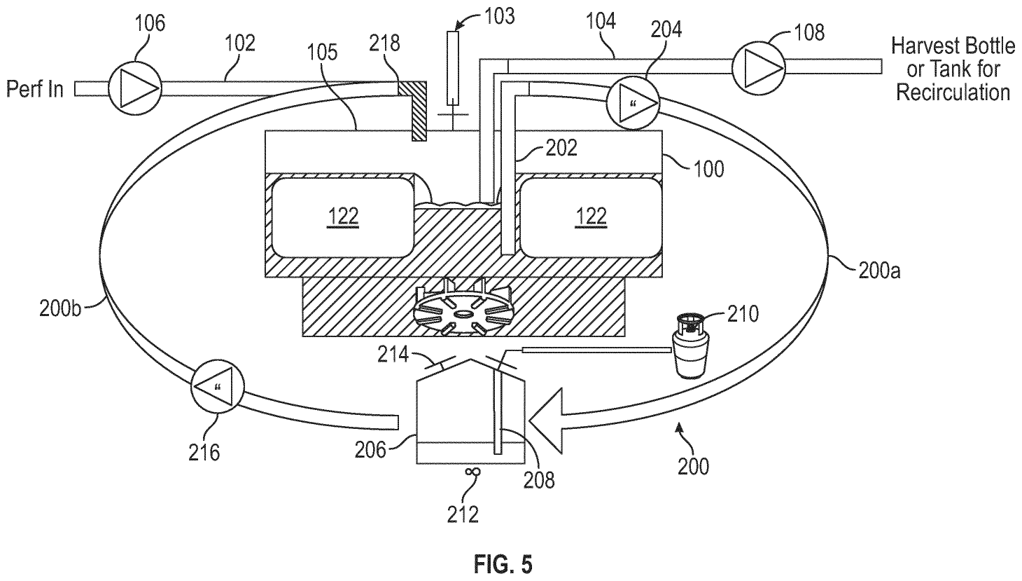

An alternate embodiment is shown in FIG. 6. In this version, a loop 300 is again external to the bioreactor 100, associated with inlet conduit 102 (e.g., a deep tube (as a drain or sampling line) or a tubing drawing in media at the air/liquid interface) and outlet conduit 104 for transmitting liquid to and from the bioreactor, respectively. This loop 300 may be associated with a suitable pump 302. A gas injector in the form of an inlet line 304 may deliver or inject gas (e.g., oxygen or air) directly to the loop 300.

An optional static mixer 306 may also be located downstream in the flow path defined by the loop 300 for mixing the injected gas with the liquid. This mixer 306 may take the form of a zig-zag system 308 (FIG. 6A) or a reducer 310 and enhancer 312 placed in series in the loop 300. In any case, the static mixer 306 passively creates turbulence to improve gas/liquid mixing within the loop 300 and helps to ensure better gas transfer efficiency.

In this or the previous embodiment, an optional sensor, such as dissolved oxygen (e.g., an inline flowcell or one located inside bioreactor 100) sensor 314, may also be used to provide feedback. This feedback may be used to control the flowrate via pump 302 or to control the gas injection rate by controlling a gas flow meter 316 associated with the gas injector, thereby ensuring the desired level of gas transfer is achieved. This approach avoids the need for dedicated input and output lines, as well as the need for an external vessel and an associated sparger, and also avoids the need for a dynamic mixer (but any or all of these features may be utilized, if desired).

Gas transfer, such as oxygenation, may occur in the gas inlet conduit 102 and the static mixer 306, as well as in all lines or tubing after the gas inlet. Gas may then be exhausted when the liquid containing gas is returned back to the bioreactor 100. For example, the liquid culture media may fall into bioreactor 100 while the gas goes into the bioreactor headspace (which may have an air vent line, as noted previously).

The above techniques may be optionally applied to the bioreactor 100, including possibly in a retrofit situation. The bioreactor 100 may or may not have an separate oxygenation system (i.e., headspace, falling film, sparger, etc.), and the external loop 200, 300 may serve as the source of oxygen or it may serve as a supplement added to the bioreactor as a “boost” when certain triggers or events occur or inputs are received. Again, the dedicated nature of the external loop 200, 300 allows for it to be used on demand without impacting the flow of liquid media to or from the bioreactor 100, such as media source and harvest vessels, or second external loop associated with a recirculation tank.

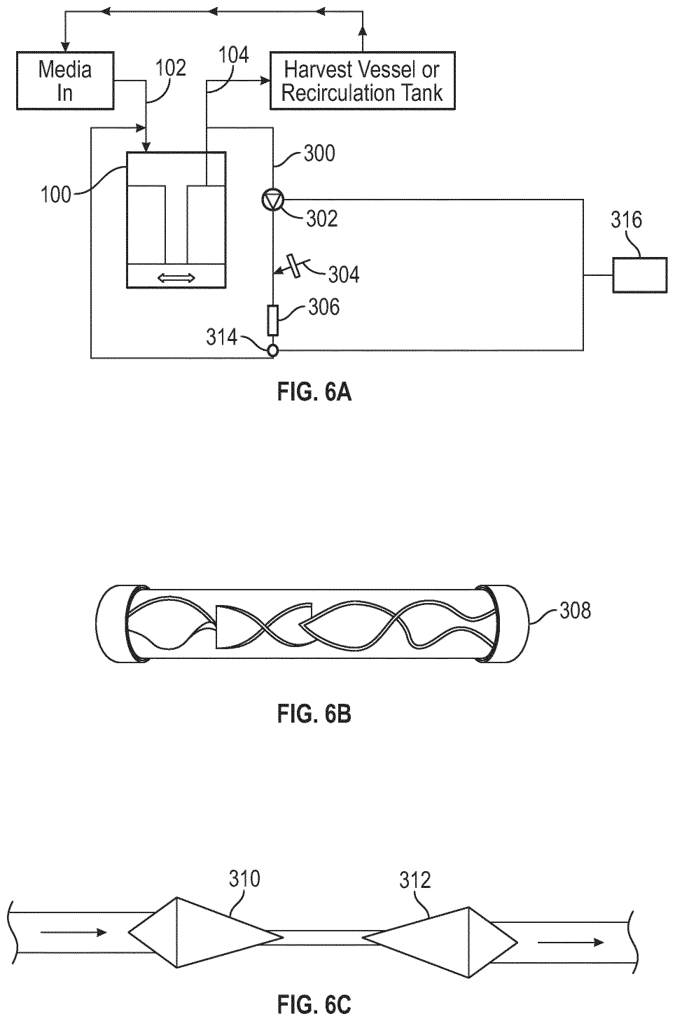

As illustrated schematically in FIG. 7, an output signal may be obtained from a sensor 400 associated with the bioreactor 100 or the external loop 200, 300, such as a dissolved oxygen sensor. Such output signal and its associated data may be processed by a controller 500, which may be adapted to control when the bioreactor 100 is in need of and thus receives a boost of gas-enhanced (e.g., oxygenated) media by delivering a portion of the liquid media therein to the external loop 200, 300. The controller 500 may activate one or more components 600, such one or more of gas injector 208, 304 and/or pumps 204, 216, 302 to control flow of liquid to the external loop 200, 300, to achieve enhanced gas transfer.

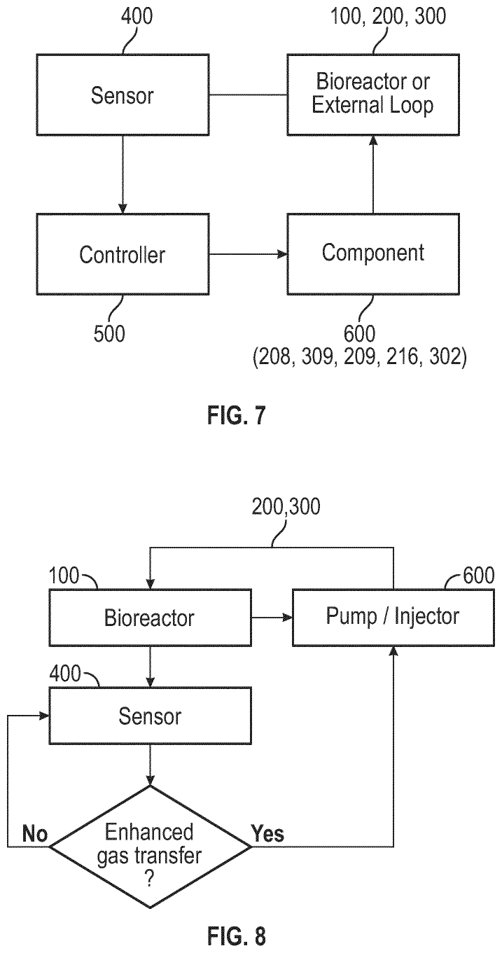

The liquid from the bioreactor 100 may be delivered to the external loop 200, 300 automatically, including when perfusion begins. Alternatively, the liquid from the bioreactor 100 may be delivered to the external loop only when a need or demand for enhanced gas transfer exists. This may be determined via a sensor 400, as shown in the flowchart of FIG. 8. In this alternative case, a portion of media flows from bioreactor 100 through the loop 200, 300 via suitable components 600 (e.g., one or more of gas injector 208, 304 and/or pumps 204, 216, 302 associated with loop 200, 300 or external vessel) only when there exists a demand for oxygen in the bioreactor 100 (such as via controller 500 programmed for this purpose, such as to maintain a certain level of oxygenation or the like). This real-time “on demand” approach provides substantial flexibility in the delivery and customization of process controls, particularly in relation to achieving enhanced gas transfer and promoting optimal cell growth conditions.

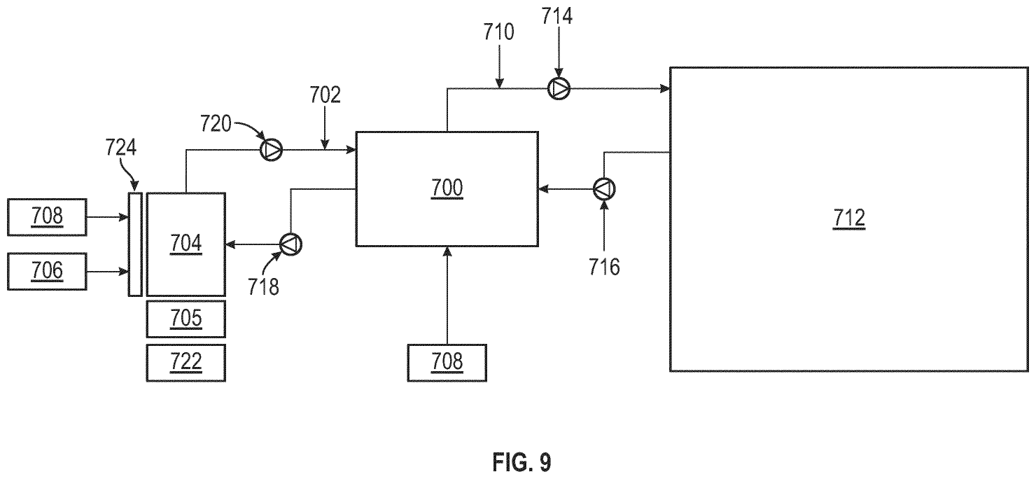

These approaches for enhanced oxygenation may also be used in connection with the use of a gas exchanger, such as a falling film, in the bioreactor 100 to further enhance the gas transfer. This may be in addition to the gas injector associated with the external vessel or loop. As also noted above, a sparger may also be used in the bioreactor 100 to further enhance the gas exchange afforded by the other proposed measures. Furthermore, suitable tubing (e.g., silicone or C-FLEX tubing) may be used to form the external loop 200, 300 to allow for additional gas (e.g., CO2) to permeate from the liquid, including in the arrangements shown in FIG. 6 (as well as FIG. 9). The external loop 200, 300 may also be used for during inoculation and/or during cell culture phases, depending on the application.

Turning now to FIG. 9, a bioreactor 700 associated with a first external loop 702 and a vessel 704 may be used for realizing enhanced gas transfer to the media (and, thus, cells) and/or transfection of such cells. Specifically, this vessel 704 or loop 702 may be associated with a gas injector 706, as noted previously. The vessel 704 may also include an agitator 705, such as an internal magnetic stir bar, impeller, or the like.

A second vessel 708 including a transfection material (e.g., transfection agent, or mixture) may be provided, which may be associated with the bioreactor 700 for delivering a transfection material thereto. While both are shown in FIG. 9 for purposes of illustration, the second vessel could additionally or alternatively be associated with the loop 702 or the vessel 704. The transfection material may comprise, for example, cationic polymers such as DEAE-dextran or polyethylenimine (PEI) or lipofectamine, and may be in the form of a transfection (lipidic) mix or mixture whereby a transfection agent is mixed with DNA (sometimes also RNA). The transfection process may comprise chemical-based transfection, such as precipitation of phosphate using calcium, lipofection (or liposome transfection), the Fugene transfection reagent, or dendrimers (highly ordered, branched polymeric molecules). In addition to transfection reagent delivery, this second vessel 708 may be used to deliver any other molecules/complexes used for gene editing (e.g. CRISPR-Cas9), which materials shall also be considered “transfection material” for purposes of this disclosure.

A second external loop 710 may be associated with a media vessel or tank 712. This loop 710 may be independent and separated from loop 702, and may be used for supplying media to the bioreactor 700 in association with pumps 714, 716. Pumps 718, 720 are also associated with the first external loop 702 for delivering fluid to or from the bioreactor 700. Alternatively, the media introduction may be by way of perfusion.

Thus, using this arrangement, the bioreactor 700 may benefit from enhanced gas transfer as the result of the use of the gas injector 706 during cell culturing, if desired (but this is considered optional). When cell transfection is to be achieved, the external loop 702 and vessel 704 may be used to receive an excess volume of liquid from the bioreactor 700 via pumps 718, 720 in connection with the addition of the transfection material (agent or mix) to the liquid, such as by introduction of the transfection material as a transfection mix directly to the bioreactor 700 (but it could also be done via loop 702 or vessel 704).

The pumps 718, 720 may be low shear pumps, such as peristaltic low shear pumps (e.g., a Watson Marlow disposable version), centrifuges (e.g., Levitronix Puralev), membrane pumps (e.g., Quatraflow disposable pump), a traditional peristaltic pump operated at a low rotational speed with large tubing to reduce the shear, or any combination of the foregoing. Use of such low shear pumps avoids damaging the transfection material (e.g., plasmid DNA), which may be shear, temperature, concentration, time and pH sensitive. By pumping liquid through the corresponding loop 702, concentration of the DNA/plasmid complex in the bioreactor 700 is also prevented.

Circulation of media to the bioreactor 700 from the media tank 712 may also be halted during the transfection process to allow for a stable pH to be achieved, and all gas transfer or oxygenation may also be halted to avoid subjecting the transfection material to bubbles (e.g., circulation of media is stopped to provide enough time for the transfection mix to interact with the cells, and pH regulation is stopped during transfection to avoid interaction of the mix with NaOH). This also avoids the need for concentrating the transfection mix for accommodation in the limited volume of the bioreactor, and a potential risk of precipitation of DNA/RNA and toxicity of the free transfecting agent.

In the case of using a low-shear centrifugal pump, the external loop 702 for the transfection should be placed lower than the bioreactor 700 to allow for priming of the pump. Likewise, the external loop 702 may be connected to the drain line of the bioreactor 700 to prevent bubbles creation, which may result from drawing fluid at the interface between the gas and liquid in the bioreactor.

In order to maintain the volume of the external loop 702 stable in view of the inability of inlet and outlet pumps to cause fluid flow at exactly the same rate, a sensor 722 may be used, such as in the form of a load cell, weight sensor, scale, balance, etc. Additionally or alternatively, a conductivity/capacitance level sensor 724 may be associated with the external loop 702 and, in particular, the vessel 704 forming a part of it to detect the level of liquid therein. The outlet pump (e.g., pump 720 in FIG. 9) may be controlled by one or more of these sensors 722, 724 to control the volume/level of the external loop 702. This approach could also be applied to the FIG. 5 embodiment.

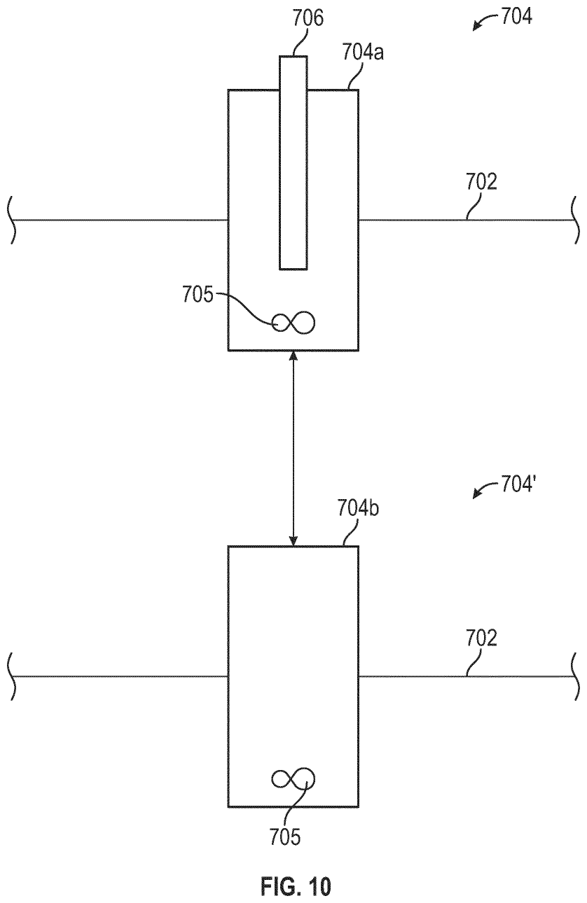

It is likewise an option to use two different vessels in place of a single vessel 704. For example, as shown in FIG. 10, a first vessel 704a for use as the external vessel 704 may be used when enhanced gas transfer is desired, and may be associated with a gas injector 706 (e.g., sparger), as well as an agitator 705. Once the target cell growth is achieved and performing a transfection step is specified, the first vessel 704a may be disconnected from the first external loop 702, and a second vessel 704b connected in its place as the external vessel 704′ for use in performing transfection, which second vessel may include an agitator but no sparger. In both cases, low-shear pumps (not shown in FIG. 10) may be used in connection with the external loop 702 to avoid causing shear stress on the transfecting material, but still conveying the liquid for purposes of the enhanced gas transfer. The first and second interchangeable vessels may be made of disposable materials.

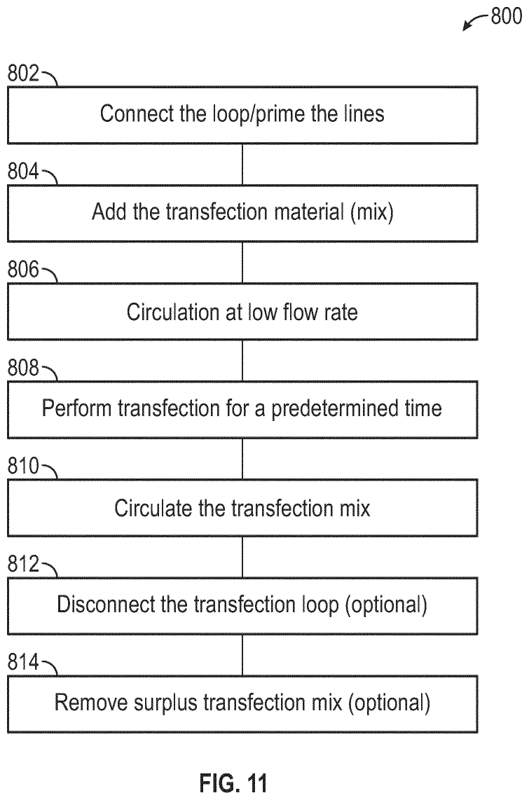

Turning to FIG. 11, a flowchart is provided to illustrate the steps associated with a process 800 of using an external loop for transfection (which may be achieved without performing any gas enhancement). The first step 802 is to connect the loop to the bioreactor, if not already present (that is, if it was not used for enhanced gas transfer, such as in the FIG. 5, 6 or 9 embodiments) and prime the lines of the loop.

The process 800 may next involve the step 804 of adding the transfection material, which may be part of a separately prepared transfection mix. Following this addition, the step 806 of circulation at a low flow rate (e.g., 0.1-5 L/min, and as an example 1-2 L/min for a bioreactor having a surface area of 600 m². The next step 808 is transfection for a period of time (e.g., 10 min to 4 h or longer), and also possibly the step 810 of circulating the transfection mix few times (e.g., if the loop is 40 L, the liquid may be circulated at 2 L/min).

Optionally, the step 812 of disconnecting the transfection loop may be performed, along with the step 814 of perfusing the liquid media inside the bioreactor to wash out the surplus of transfecting mix (potentially toxic for the cells). Multiple transfection steps may also be taken accommodating different compositions of the transfection mix (e.g., plasmids added at different times).

Aside from the ability to control the volume of the transfection loop and to provide a homogeneous mixture, the above approach to transfection via an external loop also potentially allows for “one step” transfection (but transfecting could be done in two steps if the volume is too large, but this is more complex and potentially less efficient). Furthermore, as noted above, the disclosed techniques allow for removal of the transfection mixture at the end of the process (i.e., to remove any free toxic transfecting material) by removing the external loop and perfusing the bioreactor with fresh liquid media.

Summarizing, this disclosure may related to any or all of the following items in any combination:

1. An apparatus for culturing and transfecting cells using a liquid, comprising:
a bioreactor including a cell culture bed for receiving the liquid; and
a first external loop connected to the bioreactor for circulating the liquid;
a first vessel associated with the external loop for receiving the liquid;
a gas injector for injecting a gas into the liquid; and
a second vessel for providing a transfection material to the liquid.
   2. The apparatus of item 1, wherein the second vessel is adapted for delivering the transfection material directly to the bioreactor.
   3. The apparatus of item 1 or item 2, wherein the second vessel is adapted for delivering the transfection material or other reagents for gene editing directly to the external loop.
   4. The apparatus of any of items 1-3, wherein the gas injector is adapted for delivering gas to the first vessel.
   5. The apparatus of any of items 1-4, wherein the gas injector is adapted for delivering gas to the first external loop.
   6. The apparatus of any of items 1-5, further including a second external loop for circulating liquid from a liquid media supply tank to the bioreactor.
   7. The apparatus of any of items 1-6, wherein the bioreactor further includes a sparger.
   8. The apparatus of any of items 1-7, wherein the bioreactor is adapted for forming a falling liquid to promote gas exchange therein.
   9. The apparatus of any of items 1-8, wherein the bioreactor, the first vessel, and the second vessel each include an agitator.
   10. The apparatus of any of items 1-9, wherein the first external loop includes one or more pumps.
   11. The apparatus of item 10, wherein the one or more pumps comprise one or more low-shear pumps.
   12. The apparatus of any of items 1-11, further including a sensor for sensing an amount of gas in the liquid, the sensor generating an output signal for use in regulating one or more pumps for pumping liquid through the first external loop or regulating the gas injector for injecting gas into the liquid.
   13. The apparatus of item 12, wherein the sensor is adapted for sensing the amount of gas within the liquid in the bioreactor.
   14. The apparatus of item 12, wherein the sensor comprises a dissolved oxygen sensor.
   15. The apparatus of any of items 1-14, wherein the first external loop includes a mixer.
   16. The apparatus of any of items 1-15, wherein the cell culture bed comprises a structured fixed bed, a spiral bed, or a 3-D printed monolithic matrix.
   17. The apparatus of item 1-16, wherein the transfection material is a component of a transfecting mixture added to the liquid.
   18. An apparatus for culturing and transfecting cells using a liquid, comprising:
a bioreactor including a cell culture bed for receiving the liquid;
a first external loop connected to the bioreactor and adapted for circulating the liquid via one or more low shear pumps;
a gas injector for injecting a gas into the liquid; and
a first vessel for providing a transfection material to the liquid.
   19. The apparatus of item 18, wherein the first vessel is adapted for delivering the transfection material directly to the bioreactor.
   20. The apparatus of item 18, wherein the first vessel is adapted for delivering the transfection material directly to the first external loop.
   21. The apparatus of any of items 18-20, wherein the gas injector is adapted for delivering gas to the external loop.
   22. The apparatus of item 18-20, wherein the gas injector is adapted for delivering gas to a second vessel connected to the external loop.
   23. The apparatus of any of items 18-22, further including a second external loop for circulating liquid from a tank to the bioreactor.
   24. The apparatus of any of items 18-23, wherein the bioreactor further includes a sparger.
   25. The apparatus of any of items 18-24, wherein the bioreactor is adapted for forming a falling liquid to promote gas exchange therein.
   26. The apparatus of any of items 18-25, wherein the bioreactor and the vessel each include an agitator.
   27. The apparatus of any of items 18-26, further including a sensor for sensing an amount of gas in the liquid, the sensor generating an output signal for use in regulating the one or more low shear pumps for pumping liquid through the first external loop or regulating the gas injector.
   28. The apparatus of item 27, wherein the sensor is adapted for sensing the amount of gas within the liquid in the bioreactor.
   29 The apparatus of item 27 or item 28, wherein the sensor comprises a dissolved oxygen sensor.
   30. The apparatus of any of items 18-29, wherein the first external loop includes a mixer.
   31. The apparatus of any of items 18-29, wherein the cell culture bed comprises a structured fixed bed, a spiral bed, or a 3-D printed monolithic matrix.
   32. An apparatus for culturing cells using a liquid, comprising:
a bioreactor including a cell culture bed for receiving the liquid; and
a first external loop connected to the bioreactor and adapted for circulating the liquid, the first external loop adapted for introducing a gas into the liquid therein or for circulating a transfection material in the liquid therein to the bioreactor; and
a media vessel for supplying liquid to the bioreactor independent of the first external loop.
   33. The apparatus of item 32, wherein first external loop comprises a gas injector.
   34. The apparatus of item 33, wherein the gas injector comprises a sparger associated with a vessel, the vessel optionally associated with a mixer.
   35. The apparatus of item 34, wherein the gas injector comprises a gas line for supplying the gas directly to the first external loop.
   36. The apparatus of any of items 32-35, wherein the first external loop includes one or more pumps, and optionally a second external loop in fluid communication with the bioreactor and the media vessel, the second external loop including one or more pumps.
   37. The apparatus of any of items 32-36, wherein the first external loop includes one or more low shear pumps, and further including a vessel for introducing a transfection material to the cell culture bed.
   38. The apparatus of any of items 32-37, further including a sensor for sensing an amount of gas in the liquid, the sensor generating an output signal for use in regulating one or more pumps for pumping liquid through the first external loop or the gas injector.
   39. The apparatus of any of items 32-37, wherein the first external loop includes a mixer.
   40. The apparatus of any of items 32-39, wherein the cell culture bed comprises a structured fixed bed, a spiral bed, or a 3-D printed monolithic matrix.
   41. An apparatus for culturing cells using a liquid, comprising:
a bioreactor including a cell culture bed for receiving the liquid
at least one gas sensor for sensing an amount of gas in the liquid;
an external loop connected to the bioreactor, the external loop associated with a pump for circulating the liquid and a gas injector for injecting a gas into the liquid; and
a controller for controlling the pump, the gas injector, or both, based on the output of the at least one gas sensor.
   42. The apparatus of item 41, wherein the gas injector comprises a sparger associated with a vessel, optionally associated with a mixer.
   43. The apparatus of item 41, wherein the gas injector comprises a gas line for supplying the gas directly to the external loop.
   44. The apparatus of any of items 41-43, wherein the external loop includes a plurality of pumps.
   45. The apparatus of any of items 41-43, wherein the external loop is connected to an inlet and an outlet of the bioreactor.
   46. The apparatus of any of items 41-43, wherein the external loop includes a static mixer.
   47. The apparatus of any of items 41-43, wherein the cell culture bed comprises a structured fixed bed, a spiral bed, or a 3-D printed monolithic matrix.
   48. An apparatus for culturing cells using a liquid, comprising:
a bioreactor including a cell culture bed for receiving the liquid, the bioreactor adapted for promoting gas exchange with the liquid therein;
an external loop connected to the bioreactor, the external loop associated with a pump for circulating the liquid; and
a gas injector for injecting a gas into the liquid.
   49. The apparatus of item 48, wherein the gas injector injects the gas into the liquid in the external loop.
   50. The apparatus of item 48 wherein the gas injector injects the gas into the liquid in an external vessel associated with the external loop.
   51. The apparatus of any of items 48-50, wherein the bioreactor is adapted for creating a falling film of liquid in a headspace thereof.
   52. A method for culturing and transfecting cells using a bioreactor including a cell culture bed for receiving a liquid media from a media source, comprising:
introducing a transfection material to the liquid media; and
circulating the liquid media including the transfection material via an external loop connected to the bioreactor and separate from the media source.
   53. The method of item 52, wherein the introducing step comprises introducing a transfection mix including the transfection material to the bioreactor prior to the circulating step.
   54. The method of item 53, wherein the introducing step comprises introducing the transfection material to the external loop.
   55. The method of any of items 52-54, further including the step of halting any delivery of liquid media from the media source during the introducing and circulating steps.
   56. The method of any of items 52-54, further including the step of injecting gas into the liquid via the external loop prior to the introducing step.
   57. The method of any of items 52-56, wherein the gas injecting step comprises injecting the gas into a vessel connected to the external loop.
   58. The method of any of items 52-56, wherein the gas injecting step comprises injecting the gas directly to the external loop.
   59. The method of any of items 52-58, further including the step of connecting the external loop to the bioreactor after cell culturing is complete and prior to the introducing step.
   60. A method for regulating the culturing of cells using a bioreactor including a cell culture bed for receiving a liquid from a media source, comprising:
injecting a gas into a liquid in an external loop connected to the bioreactor and separate from the media source.
   61. The method of item 60, wherein the gas injecting step comprises injecting the gas into a vessel connected to the external loop.
   62. The method of item 60, wherein the gas injecting step comprises injecting the gas directly to the external loop.
   63. The method of any of items 60-62, further including the following steps:
halting the gas injecting step;
introducing a transfection material to the liquid; and
circulating the liquid including the transfection material via the external loop.
   64. The method of any of items 60-63, further including the step of regulating the injecting of the gas based on a sensed parameter of the liquid.
   65. The method of any of items 60-64, further including the step of regulating a flow rate of the liquid in the external loop based on a sensed parameter.
   66. A method for regulating the culturing of cells using a bioreactor including a cell culture bed for receiving a liquid from a media source, comprising:
sensing a parameter of the liquid; and
based on the sensed parameter, regulating the delivery of liquid within an external loop connected to the bioreactor, the external loop adapted for injecting gas to the liquid, regulating the injecting of gas to the external loop, or both.
   67. The method of item 66, further including the step of injecting gas into the liquid via the external loop.
   68. The method of item 66, further including the step of injecting the gas into a vessel connected to the external loop.
   69. The method of any of items 66-68, wherein the sensing step comprises sensing dissolved oxygen as the liquid.
   70. The method of item 66, wherein the sensing step is completed by a sensor in the bioreactor.
   71. The method of item 66, wherein the sensing step is completed by a sensor in the external loop.
   72. A method of bioprocessing, comprising:
culturing cells in a bioreactor including a liquid;
during the culturing step, injecting gas into the liquid in an external loop in fluid communication with the bioreactor;
transfecting the cells by adding a transfection material to the liquid; and
delivering a portion of the liquid including the transfection material to the external loop.
   73. The method of item 72, wherein the external loop includes a vessel, and the injecting step comprises injecting gas into the vessel.
   74. The method of item 72, wherein the external loop includes a vessel, and the delivering step comprises delivering the portion of the liquid including the transfection material to the external loop.
   75. The method of item 72, wherein the transfecting step comprises adding the transfection material to the bioreactor.
   76. The method of any of items 72-75, further including the steps of:
sensing a parameter of the liquid; and
based on the sensed parameter, regulating the delivery of liquid to the external loop or the injecting of gas to the external loop.
   77. The method of any of items 72-76, wherein the transfecting step comprises circulating the transfecting material within the bioreactor for a predetermined time.
   78. The method of any of items 72-77, further including the step of halting the gas injecting step prior to and during the transfecting step.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

While preferred embodiments have been shown and described herein, it will be obvious to those skilled in the art that such embodiments are provided by way of example only. Numerous variations, changes, and substitutions will now occur to those skilled in the art without departing from the invention. For example, while the bioreactor is sometimes shown in a vertical orientation, it could be used in any orientation. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1.-78. (canceled)

79. An apparatus for culturing cells using a liquid, comprising:
a bioreactor including a cell culture bed for receiving the liquid;
a first external loop connected to the bioreactor, the first external loop associated with a pump for circulating the liquid; and
a gas injector for injecting a gas into the liquid.

80. The apparatus of claim 79, wherein the gas injector injects the gas into the liquid in the first external loop or the gas injector injects the gas into the liquid in an external vessel associated with the first external loop.

81. The apparatus of claim 79, wherein the bioreactor is adapted for creating a falling film of liquid.

82. The apparatus of claim 79, further including a second vessel for providing a transfection material to the liquid.

83. The apparatus of claim 82, wherein the second vessel is adapted for delivering the transfection material directly to the bioreactor or for delivering the transfection material or other reagents for gene editing directly to the first external loop.

84. The apparatus of claim 79, further including a sensor for sensing an amount of gas in the liquid, the sensor generating an output signal for use in regulating the pump for pumping liquid through the first external loop or regulating the gas injector for injecting gas into the liquid.

85. The apparatus of claim 79, further including a second external loop for circulating liquid from a liquid media supply tank to the bioreactor

86. An apparatus for culturing and transfecting cells using a liquid, comprising:
a bioreactor including a cell culture bed for receiving the liquid; and
a first external loop connected to the bioreactor for circulating the liquid;
a first vessel associated with the external loop for receiving the liquid;
a gas injector for injecting a gas into the liquid; and
a second vessel for providing a transfection material to the liquid.

87. The apparatus of claim 86, wherein the second vessel is adapted for delivering the transfection material directly to the bioreactor.

88. The apparatus of claim 86, wherein the second vessel is adapted for delivering the transfection material directly to the first external loop.

89. The apparatus of claim 86, wherein the gas injector is adapted for delivering gas to the first vessel.

90. The apparatus of claim 86, wherein the gas injector is adapted for delivering gas to the first external loop.

91. The apparatus of claim 86, further including a second external loop for circulating liquid from a liquid media supply tank to the bioreactor.

92. The apparatus of claim 86, wherein the bioreactor is adapted for forming a falling liquid to promote gas exchange therein.

93. The apparatus of claim 86, further including a sensor for sensing an amount of gas in the liquid, the sensor generating an output signal for use in regulating one or more pumps for pumping liquid through the first external loop or regulating the gas injector for injecting gas into the liquid.

94. An apparatus for culturing cells using a liquid, comprising:
a bioreactor including a cell culture bed for receiving the liquid; and
a first external loop connected to the bioreactor and adapted for circulating the liquid, the first external loop adapted for introducing a gas into the liquid therein or for circulating a transfection material in the liquid therein to the bioreactor; and
a media vessel for supplying the liquid to the bioreactor independent of the first external loop.

95. The apparatus of claim 94 wherein first external loop comprises a gas injector.

96. The apparatus of claim 94, wherein the gas injector comprises a sparger associated with a vessel connected to the first external loop or a gas line for supplying the gas directly to the first external loop.

97. The apparatus of claim 94, further including a sensor for sensing an amount of gas in the liquid, the sensor generating an output signal for use in regulating one or more pumps for pumping liquid through the first external loop or the gas injector.

98. The apparatus of claim 94, further including a second external loop for circulating liquid from a liquid media supply tank to the bioreactor
